(19) United States
(12) Patent Application Publication
(10) Pub. No.: US 2006/0167026 A1
(43) Pub. Date: Jul. 27, 2006

(54) Antipsychotic molecular-targeting epithelial growth factor receptor

(75) Inventors: Hiroyuki Nawa, Niigata (JP); Makoto Mizuno, Niigata (JP)

Correspondence Address: BURR & BROWN, PO BOX 7068, SYRACUSE, NY, 13261-7068, US

(73) Assignee: Hiroyuki Nawa, Niigata-shi (JP)

(21) Appl. No.: 10/540,989

(22) PCT Filed: Jan. 5, 2004
(86) PCT No.: PCT/JP04/00002

§ 371 Date: Jan. 3, 2006

(30) Foreign Application Priority Data
Jan. 6, 2003 (JP) 2003034396

(51) Int. Cl. A61K 31/517

(52) U.S. Cl. 514/266.4

(57) ABSTRACT

The purpose of this invention is to provide an agent useful for prevention and/or treatment of psychosis, schizophrenia and cognitive impairments. To solve this problem, this invention provides epidermal growth factor receptor inhibitors as therapeutic agents for psychosis, schizophrenia and cognitive impairments.

### 1. FIELD OF THE INVENTION

This invention relates to novel antipsychotic drugs that are beneficial for the treatment of psychosis. In more detail, this invention relates to the use of epidermal growth factor receptor inhibitors for prevention or treatment of schizophrenia and the use of epidermal growth factor receptor inhibitors for prevention or treatment of cognitive impairments.

### 2. DESCRIPTION OF RELATED ARTS

0.7-1.0% of human population are suffering from schizophrenia. There are more than a few hundreds of patients with schizophrenia in Japan, which is one of very serious and problematic psychiatric disorders with chronic patients. Major symptoms of this disorder include a large variety of psychiatric impairments, which consist of the positive symptoms such as delusion, visual hallucination, auditory hallucination, the cognitive defects such as sensory abnormalities, and the negative symptoms of social withdrawal and depression. At present, we understand neither the causative etiology of schizophrenia nor biological basis of its neuropathology.

Schizophrenia has an onset between adolescence and prime stages of human life and persists chronically to put many difficulties on its patients in respect of perception, cerebration, emotion and behavior. Psychiatric symptoms of this disorder are classified into the three categories of positive symptoms (delusion, hallucination, lower thinking ability, bizarre behaviors) negative symptoms (emotional loss, anhedonia, asociality) and cognitive impairments (working memory deficits, aphasia, attention deficit). Each patient displays a distinct set of these symptoms. From social point of view, it has been hoped to establish the system of consistent and comprehensive treatments including early diagnosis, medication and prevention of schizophrenia relapse because of the above specificity of its psychopathology. However, it is relatively difficult to cure this disease completely.

Until now, the agents that compete the neurotransmitter, dopamine, are suggested to be beneficial to treat the positive symptoms of schizophrenia. Typical antipsychotics of first choice such as haloperidol and chlorpromazine have a therapeutic effect on the positive symptoms of schizophrenia with their strong blockade of dopamine D2 receptors. In contrast, the actions of these drugs on the negative symptoms and cognitive defects are quite limited. In addition, it is essential for schizophrenia patents to take these antipsychotics for longer time periods, which result in the side effects. These side effects are called extrapyramidal symptoms including Parkinson symptoms, akathisia, dyskinesia etc, and reported to be problematic in the non-patent literature #1.

Recently, a new series of antipsychotic drugs are referred to as atypical antipsychitics such as clozapine and risperidone, which compete both dopamine and serotonin and relatively do not induce the extrapyramidal side effects (non-patent literature #2). Although the atypical antipsychotic drugs are beneficial for the treatment of the negative symptoms, clozapine has a serious risk to produce the side effect of agranulosis. In addition, the high doses of risperidone produce the extrapyramidal side effects as typical antipsychotic drugs do.

To aim at curing the variety of the psychotic pathology of schizophrenia, chemical derivatives of phenothiazine, thioxanthene, bromophenone and benzuamide including the above compounds have been developed and applied to patents until now. There are very limited cases in which these many antipsychotics led to the complete recovery from schizophrenia. Accordingly, there has been demand on the new antipsychotics that do not target the antagonism of dopamine or serotonin.

Epidermal growth factor is involved in cell proliferation, in particular cancer growth. Thus, a variety of binding inhibitors of epidermal growth factor to the receptors as well as those of kinase blockers for epidermal growth factor receptors have been developed by pharmaceutical companies as anti-cancer drugs (non-patent literature #3).

On the other hand, little is known about the direct relation between the onset/pathology of schizophrenia and the hyperactivity of growth factors/neurotrophic factors regulating normal brain development. Recently, the inventors verified the involvement of some of these factors in schizophrenia and reported the results in the non-patent literature #4. In parallel, the patent application has been filed, which claims the use of these factors as a diagnosis marker of schizophrenia (patent literature #1).

In fact, the proceeding researches of the present inventor demonstrate that protein content of epidermal growth factor is decreased and the expression of its receptors is conversely increased in the brain of schizophrenic patients (non-patent literature #5). Among many proteins, the increase in the expression of epidermal growth factor receptors is most remarkable in the prefrontal cortex and striatum, which are the brain regions responsible for the cognitive function in human.

These facts suggest the possibility that some of schizophrenia pathology involve alteration in the activity of epidermal growth factor receptors. The evidence has, however, verified neither the therapeutic target being epidermal growth factor receptors nor the therapeutic effectiveness of activity blockers for epidermal growth factor receptors in the prevention or medication of schizophrenia. This explanation is supported by the following reasons: Epidermal growth factor receptor is not a sole protein whose expression is altered in schizophrenia. The alteration in the protein expression may be a result of the disease onset. Even if it has a causative role, it is uncertain whether the agents acting on epidermal growth factor receptors have not enough preventive or therapeutic effects. Alternatively, it is also possible that the protein levels change without reflecting the direct cause or result of this disease.

In general, schizophrenia is thought to represent a group of various psychiatric syndromes affected by heredity and environmental factors, although its etiology is unknown. The whole genome-wide association study of this disease reports that multiples genes including those located on chromosomes, 6p22, 8p21-22, 22q12-13, are associated with schizophrenia and indicates correlation between multiple genes and this disease (non-patent literature #6).

The chromosomal region for epidermal growth factor, 4q25-27, is also a candidate locus associating with schizophrenia but the association is suggested to be lower than the other regions reported as above (non-patent literature #7).

The most popular hypothesis for schizophrenia is the hypothesis of abnormal brain development. In addition to the multiple gene abnormalities as described above, environmental effects, such as viral infection and obstetric complications, impair brain development in human being, leading to disorder the cognitive brain function. In fact, animal modeling revel that maternal administration to influenza virus (non-patent literature #8) and bacterial toxin (non-patent literature #9), and neonatal exposures to interleukin-1 (non-patent literature #2), leukemia inhibitory factor (LIF) (patent literature #3) and epidermal growth factor (patent literature #1) impair brain development, leading to gradual cognitive and behavioral disturbances of the offspring during development

In the experiment, influenza virus is administered to impair brain development. As these factors are given with injection to maternal animals or by neonatal exposure, their actions are transient. The continuous administration thereafter of the factors is not necessary, however, to induce the cognitive impairments. Accordingly, the animal modeling studies failed to reveal that any of these factors would be causes or specific targets for the therapeutic application.

These results demonstrated that multiple maternal and perinatal environments, as well as multiple genetic components, induce later abnormalities in brain function. Accordingly, cognitive dysfunctions including schizophrenia in human involve multiple causes, risks and mediators and are not ascribed to a single factor.

It has remained to be clarified whether inhibitors for epidermal growth factor receptors therapeutically act against schizophrenia until the present discovery of inventors described in this patent. Moreover, the examples of therapeutic application of inhibitors for epidermal growth factor and blockers for epidermal growth factor receptors for psychiatric diseases have not been reported yet.

<non-patent literature #1>

Casey D. E. et al.; J. Clinical Psychiatry 58, p 55-62 (1997).
   <non-patent literature #2>
Kapur S. et al.; Am. J. Psychiatry 153, p 466-476 (1996).
   <non-patent literature #3>
Fry, D. W. et al.; Anti-Cancer Drug Design 15, p 3-16 (2000).
   <non-patent literature #4>
Nawa H. et al.; Mol. Psychiatry 5, p 594-603 (2000).
   <non-patent literature #5>
Futamura T. et al.; Mol. Psychiatry 7, p 673-682 (2002).
   <non-patent literature #6>
Berry N. et al.; J. Psychiatry Neurosci. 28(6), p 415-429 (2003).
   <non-patent literature #7>
Paunio T. et al.; Hum. Mol. Genet 58, p 3037-3048 (2001).
   <non-patent literature #8>
Shi L. et al.; J. Neuroscience 23(1), p 297-302 (1997).
   <non-patent literature #9>
Borrell J. et al.; Neuropsychopharmacol 26(2), p 204-215 (2002).
   <Patent literature #1>
Japan Patent Application; 2000-309042
   <Patent literature #2>
Japan Patent Application; 2001-52546
   <Patent literature #1>
Japan Patent Application; 2002-382835

### SUMMARY OF THE INVENTION

The inventors have carried out the extensive investigations to resolve the above the problem and demonstrated that the agents inhibiting the action of epidermal growth factor receptors are beneficial to ameliorate the symptoms of schizophrenia and related disorders.

That is, this invention provides the following preventive and/or therapeutic agents.

(1) A preventive and/or therapeutic agent for schizophrenia containing an inhibitor of epidermal growth factor receptor as the active ingredient.

(2) The preventive and/or therapeutic agent according (1), wherein the inhibition is a competitive inhibition on binding between epidermal growth factor receptor and epidermal growth factor.

(3) A preventive and/or therapeutic agent for schizophrenia containing an inhibitor of epidermal growth factor receptor as the active ingredient.

(4) The preventive and/or therapeutic agent according to (3), wherein the inhibition is a competitive inhibition on binding between epidermal growth factor receptor and epidermal growth factor.

(5) A preventive and/or therapeutic agent for cognitive abnormalities containing an inhibitor of epidermal growth factor receptor as the active ingredient.

(6) The preventive and/or therapeutic agent according to (5), wherein the inhibition is a competitive inhibition on binding between epidermal growth factor receptor and epidermal growth factor.

(7) The preventive and/or therapeutic agent according to (1) or (3) or (5) containing a quinazoline derivative having inhibitory activity to epidermal growth factor receptor represented by the chemical formula I, a stereoisomer thereof, a pharmaceutically-acceptable salt thereof, its hydrate or its solvate as the effective ingredient, 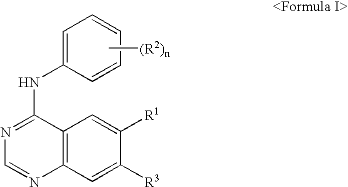
wherein; n is 1, 2 or 3 and R² is each independently halogen, trifluoromethyl, or (1-4C)alkoxy; R³ is (1-4C)alkoxy; and R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy, and, wherein any of the above-mentioned R¹ substituents comprising a CH₂ (methylene) group which is not attached to N or O atom optionally bears a hydroxy substituent on said CH₂ group.

(8) The preventive and/or therapeutic agent according to (1) or (3) or (5) containing a quinazoline derivative represented by the chemical formula II, a stereoisomer thereof, a pharmaceutically-acceptable salt thereof, its hydrate or its solvate as the effective ingredient, 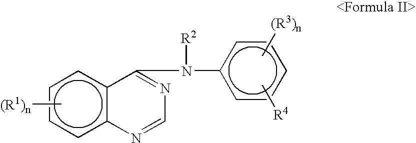
wherein m is 1, 2, or 3; R¹ is each independently selected from the group consisting of hydrogen, halo, hydroxy, amino, hydroxyamino, carboxy, (C₁-C₄)alkoxycarbonyl, nitro, guanidino, ureido, carbarnoyl, cyano, trifluoromethyl, (R⁶)₂N-carbonyl, and phenyl-W-alkyl (wherein W is selected from the group consisting of a single bond, O, S and NH); or R¹ is each independently selected from the group consisting of cyano-(C₁-C₄)-alkyl and R⁹ (wherein R⁹ is selected from the group consisting of R⁵, R⁵O, (R⁶)₂N, R⁷C(═O), R⁵ONH, A and R⁵Y; R⁵ is (C₁-C₄)alkyl; R⁶ is hydrogen or R⁵ wherein the R⁵s are the same or different; R⁷ is R¹, R⁵0 or (R⁶)₂N; A is selected from the group consisting of piperidino-, morpholino, pyrrolidino and 4-R⁶-piperazin-1-yl, imidazol-1-yl, 4-pyridon-1-yl, carboxy-(C₁-C₄)-alkyl, phenoxy, phenyl, phenylsulfanyl, (C₇-C₄)-alkenyl, (R⁶)₂—N-carbonyl-(C₁-C₄)-alkyl; and Y is selected from the group consisting of S, SO, SO₂; the alkyl moieties in R⁵, R⁵O and (R⁶)₂N are halo or R⁹ (wherein R⁹ is defined as above) and wherein the resulting groups are optionally substituted with halo or R⁹, with the proviso that a nitrogen, oxygen or sulfur atom and another heteroatom can not be attached to the same carbon atom, and with the further proviso that no more than three “R⁹” units may comprise R¹; or each R¹ is each independently selected from the group consisting of R⁵-sulfonylamono, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino (wherein R¹⁰ is selected from halo, R⁶O, (C₂-C₄)-alkanoyloxy, R⁷C(═O), and (R⁵)₂N; and wherein said benzamido or benzenesulfonylamino or phenyl or phenoxy or anilino or phenylsulfanyl substituent in R¹ may optionally bear one or two halogens, (C₁-C₄)alkyl, cyano, methansulfonyl or (C₁-C₄)-alkoxy substituents); or any two R¹s taken together with the carbons to which they are attached may comprise a 5-8 membered ring comprising at least one or two heteroatoms selected from oxygen, sulfur or nitrogen; and wherein the alkyl groups and alkyl portions of the alkoxy or alkylamino groups may be straight chained or if comprised of at least three carbons may be branched or cyclic; R² is selected from hydrogen and optionally substituted(C₁-C₆)-alkyl; n is 1 or 2 and each R³ is independently selected from hydrogen, optionally substituted (C₁-C₆)-alkyl, optionally substituted amino, halo, hydroxy, optionally substituted hydroxy; R⁴ is azido or R¹¹-ethynyl (wherein R¹¹ is selected from hydrogen, optionally substituted(C₁-C₆)alkyl, wherein the substituents are selected from the group consisting of hydrogen, amino, hydroxy, R⁵0, R⁵NH and (R⁵)₂N.

(9) The preventive and/or therapeutic agent according to (1) or (3) or (5) containing a quinazoline derivative having inhibitory activity to epidermal growth factor receptor represented by the chemical formula III, a stereoisomer thereof, a pharmaceutically-acceptable salt thereof, its hydrate or its solvate as the effective ingredient, 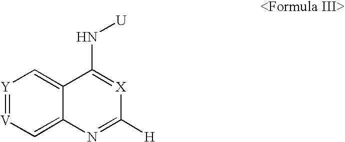
wherein X is N or CH; Y is CR¹ and V is N; or Y is N and V is CR¹; or Y is CR¹ and V is CR²; or Y is CR² and V is CR¹; R¹ represents a group CH₃SO₂CH₂CH₂NHCH₂—Ar—, (wherein Ar is selected from the group consisting of phenyl, furan, thiophene, pyrrole and thiazole, each of which may optionally be substituted by one or two halo, C₁₋₄ alkyl or C₁₋₄ alkoxy groups); R² is selected from the group consisting of hydrogen, halo, hydroxy, C₁₋₄ alkyl, C₁₋₄ alkoxy, C₁₋₄ alkylamino and di[C₁₋₄ alkyl]amino; U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl and 1H-benzotriazolyl group, substituted by an R³ group and optionally substituted by at least one R⁴ group selected independently; R³ is selected from a group consisting of benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl; or R³ represents trihalomethylbenzyl or trihalomethylbenzyloxy; or R³ represents a group of formula IV 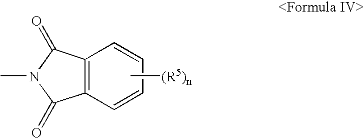
wherein each R⁵ is independently selected from the group consisting of halogen, C₁₋₄ alkyl and C₁₋₄ alkoxy; and n is 0 to 3; each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; with the proviso that the following compounds and their hydrochloride salts are excluded:
(1-Benzyl-1H-indazol-5-yl)-(6-(5-((2-methanesulphonyl-ethylamino)-methyl)-furan-2-yl)-pyrido[3,4-d]pyrimidin-4-yl-amine;
(4-Benzyloxy-phenyl)-(6-(5-((2-methanesulphonyl-ethylamino)-methyl)-furan-2-yl)-pyrido[3,4-d]pyrimidin-4-yl-amine;
(1-Benzyl-1H-indazol-5-yl)-(6-(5-((2-methanesulphonyl-ethylamino)-methyl)-furan-2-yl)-quinazolin-4-yl-amine;
(1-Benzyl-1H-indazol-5-yl)-(7-(5-((2-methanesulphonyl-ethylamino)-methyl)-furan-2-yl)-quinazolin-4-yl-amine;
(1-Benzyl-1H-indazol-5-yl)-(6-(5-((2-methanesulphonyl-ethylamino)-methyl)-1-methyl-pyrrol-2-yl)-quinazolin-4-yl-amine).

(10) The preventive and/or therapeutic agent according to (1) or (3) or (5) containing a quinazoline derivative having inhibitory activity to epidermal growth factor receptor represented by the chemical formula V, a stereoisomer thereof, a pharmaceutically-acceptable salt thereof, its hydrate or its solvate as the effective ingredient, 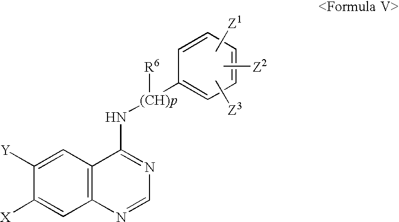
wherein X is -D-E-F and Y is —SR⁴, —OR⁴, —NHR³, or hydrogen, or X is —SR⁴, —OR⁴, —NHR³, or hydrogen, and Y is -D-E-F
D is NR²—, —O—, —CHR²—, —NR²—NH—, —NR²—O—, —CHR²—O—, —CHR²—CH₂—, —CHR²—CH₂—, NH—CHR²—, —O═CHR²—, —S—CHR²—, or D does not exist;
E is —CO—, —SO₂—, —PO(OR²)—, or —SO—;
F is —CR¹═CHR⁵—, —C≡C—R⁵—, —CR¹═C═CHR⁵;
with the proviso that when E is —SO— or —SO₂—, D is not —NH—CHR²—, or —O═CHR²;
R¹ is hydrogen, halogen, or C₁-C₆ alky1;
R², R³, and R are independently hydrogen, C₁-C₆ alky1, —(CH₂)ₙ—N-piperidiny1, —(CH₂)ₙ—N-piperaziny1,
—(CH₂)ₙ—N₁-piperaziny1 [N₄—(C₁-C₆)alky1]
—(CH₂)ₙ—N-pyrrolidy1, —(CH₂)ₙ—N-pyridiny1,
—(CH₂)ₙ—N-imidazoy1, —(CH₂)ₙ-imidazoy1
—(CH₂)ₙ—N-morpholino,
—(CH₂)ₙ—N-thiomorpholino,
—(CH₂)ₙ—N-hexahydroazepine or substituted C₁-C₆ alky1,
wherein the substituents are selected from —OH, —NH₂, or —NA-B, A and B are independently hydrogen, C₁-C₆ alkyl, —(CH₂)ₙOH, —(CH₂)ₙ-N-piperidinyl, —(CH₂)ₙ—N-piperazinyl, —(CH₂)ₙ—N₁-piperazinyl[N₄-(C₁-C₆ alkyl)], —(CH₂)ₙ—N-pyrrolidyl, —(CH₂)ₙ—N-pyridyl, —(CH₂)ₙ-imidazoyl or —(CH₂)ₙ—N-imidazoyl; Z¹, Z² or Z³ are independently hydrogen, halogen, C₁-C₆ alkyl, C₃-C₈ cycloalkyl, C₁-C₆ alkoxy, C₃-C₈ cycloalkoxy, nitro, C₁-C₆ perfluoroalkyl, hydroxy, C₁-C₆ acyloxy, —NH₂, —NH(C₁-C₆ alkyl), —N(C₁-C₆ alkyl)₂, —NH(C₃-C₈ cycloalkyl), —N(C₃-C₈ cycloalkyl)₂, hydroxymethyl, C₁-C₆ acyl, cyano, azido, C₁-C₆ thioalkyl, C₁-C₆ sulfinylalkyl, C₁-C₆ sulfonylalkyl, C₃-C₈ thiocycloalkyl, C₃-C₈ sulfinylcycloalkyl, C₃-C₈ sulfonylcycloalkyl, mercapto, C₁-C₆ alkoxycarbonyl, C₃-C₈ cycloalkoxycarbonyl, C₂-C₄ alkenyl, C₄-C₈ cycloalkenyl, or C₂-C₄ alkynyl; and R⁵ is hydrogen, halogen, C₁-C₆-perfluoroalkyl, 1,1-difluoro(C₁-C₆)alkyl, C₁-C₆alkyl, —(CH₂)ₙ—N-piperidinyl, —(CH₂)ₙ-piperazinyl, —(CH₂)ₙ-piperazinyl[N₄—(C₁-C₆)alkyl], —(CH₂)ₙ—N-pyrrolidyl, —(CH₂)ₙ-pyridinyl, —(CH₂)ₙ—N-imidazoyl, —(CH₂)ₙ—N-morpholino, —(CH₂)ₙ—N-thiomorpholino, —CH═CH₂, —CH═CH—(C₁-C₆), N-hexahydroazepine, —(CH₂)ₙNH₂, —(CH₂)ₙNH(C₁-C₆ alkyl), —(CH₂)ₙ—N(C₁-C₆ alkyl)₂, -1-oxo(C₁-C₆)alkyl, carboxy, (C₁-C₆)alkyloxycarbonyl, N—(C₁-C₆)alkylcarbamoyl, phenyl or substituted phenyl, wherein the substituted phenyl may have from one to three substituents independently selected from Z¹, Z², Z³ or a monocyclic heteroaryl group, and each C₁-C₆ alkyl group may be substituted with —OH, —NH₂ or —NAB, (wherein and B are as defined above), R⁶ is hydrogen or C₁-C₆ alkyl; and n is 1 to 4, p is 0 or 1.

(11) The preventive and/or therapeutic agent according to (1) or (3) or (5) containing a compound having inhibitory activity to epidermal growth factor receptor represented by the chemical formula VI, a stereoisomer thereof, a pharmaceutically-acceptable salt thereof, its hydrate or its solvate as the effective ingredient, 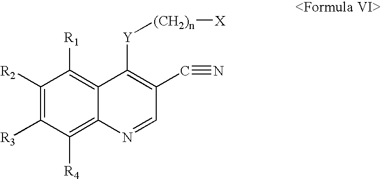
wherein X is cycloalkyl of 3 to 7 carbon atoms, which may be optionally substituted with one or more alkyl groups having 1 to 6 carbon atom; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; n is 0-1; Y is —NH—, —O—, —S—, or —NR—; R is alkyl of 1-6 carbon atoms; R₁, R₂, R₃, and R₄ are each independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 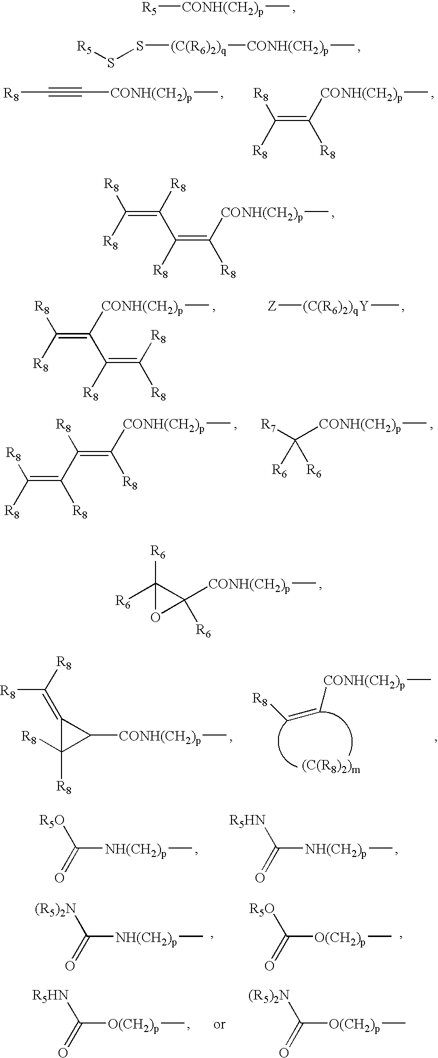
wherein, R₅ is alkyl of 1-6 carbon atoms, alkyl optionally substituted with one or more halogen atoms; phenyl, or phenyl optionally substituted with one or more halogen, alkoxy of 1-6 carbon atoms, trifluoromethyl, amino, nitro, cyano, or alkyl of 1-6 carbon atoms groups; R₆ is hydrogen, alkyl of 1-6 carbon atoms, or alkenyl of 2-6 carbon atoms; R₇ is chloro or bromo; R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl (wherein the alkyl group has 1-6 carbon atoms), piperidino-N-alkyl (wherein the alkyl group has 1-6 carbon atoms), N-alkyl-piperidino-N-alkyl (wherein either alkyl group has 1-6 carbon atoms), azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo; Z is amino, hydroxy, alkoxy of 1-6 carbon atoms, alkylamino (wherein the alkyl moiety has 1-6 carbon atoms), dialkylamino (wherein each of the alkyl moieties has 1-6 carbon atoms), morpholino, piperazino, N-alkylpiperazino (wherein the alkyl moiety has 1-6 carbon atoms), or pyrrolidino; m=1-4, q=1-3, and p=0-3; any of the substituents R₁, R₂, R₃, or R₄ that are located on contiguous carbon atoms may together be the divalent group —O—C(R₈)₂—O— (with the proviso that when Y is —NH—, R₁, R₂, R₃, and R₄ are hydrogen, and when n is 0, X is not 2-methylphenyl).

(12) The preventive and/or therapeutic agent according to (1) or (3) or (5) containing a cinnamide derivative represented by the chemical formula VII, a stereoisomer thereof, a pharmaceutically-acceptable salt thereof, its hydrate or its solvate as the effective ingredient, 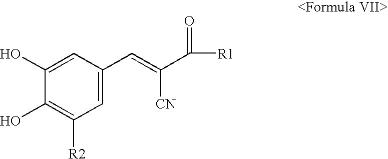
wherein R1 is preferably hydroxy, amino, alkylamino or phenyl amino group and R2 is preferably hydrogen, hydroxyl, nitro or t-butyl group.

(13) The preventive and/or therapeutic agent according to (1) or (3) or (5) containing a pyridopyrimidine derivative represented by the chemical formula VIII, a stereoisomer thereof, a pharmaceutically-acceptable salt thereof, its hydrate or its solvate as the effective ingredient, 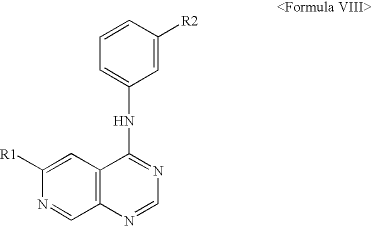
wherein R1 is preferably hydroxyl, amino, lower alkylamino, amide, alkylamide, alkenesulfinyl, or alkeneoxyamino group and R2 is preferably hydrogen or acetylene group.

(14) The preventive and/or therapeutic agent according to (1) or (3) or (5) containing a tyrosine derivative represented by the chemical formula IX, a stereoisomer thereof, a pharmaceutically-acceptable salt thereof, its hydrate or its solvate as the effective ingredient, 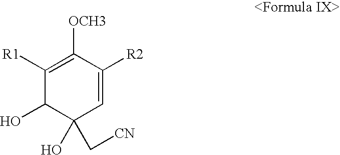
wherein R1 and R2 are preferably halogen atoms.

(15) The preventive and/or therapeutic agent according to (1) or (3) or (5) containing 4-(3-chloro-4-fluoroanilino)-7-methoxy-6-(3-morpholinopropoxy)quinazoline, a stereoisomer thereof, a pharmaceutically-acceptable salt thereof, its hydrate or its solvate as the effective ingredient.

(16) The preventive and/or therapeutic agent according to (1) or (3) or (5) containing {4-(3-bromophenyl)anilino}-6,7-diamino quinazoline, a stereoisomer thereof, a pharmaceutically-acceptable salt thereof, its hydrate or its solvate as the effective ingredient.

The above compounds that are represented with these formulas can be synthesized according to the method described in WO96/33980, WO96/30347, WO99/35146, WO97/38983, WO98/43960.

### BRIEF DESCRIPTION OF THE DRAWINGS

FIG. 1 shows a decrease in prepulse inhibition observed in rats to which EGF was administered as neonates. It is a control for FIG. 3 data.

Explanation of the marks;

open bars for normal control rats (9 weeks old), dotted bars for the rats for cognitive dysfunction (9 week old). * represents significant difference.

FIG. 2 shows a decrease in prepulse inhibition observed in rats to which EGF was administered as neonates. The rats to which EGF was administered as neonates exhibited a decrease in prepulse inhibition. It is a control for FIG. 4 data.

Explanation of the marks;

open bars for normal control rats (9 week old), dotted bars for the rats for cognitive dysfunction (9 week old). * represents significant difference.

FIG. 3 shows an ameliorative effect to decreased prepulse inhibition observed in only rats treated with EGF as neonates, by administration of the inhibitor for the activity of epidermal growth factor receptors.

Explanation of the marks;

an open bar for normal control rats receiving physiologic saline, a dotted bar for control normal receiving the compound A, a meshed bar for the model rats showing cognitive dysfunction and receiving physiologic saline, and a closed black bar for the model rats for cognitive dysfunction and receiving compound A. * represents significant difference.

FIG. 4 shows an ameliorative effect on prepulse inhibition observed in only rats to which EGF was administered as neonates, by administration of the inhibitor for the activity of epidermal growth factor receptors.

Explanation of the marks;

an open bar for normal control rats receiving physiologic saline, a dotted bar for control normal receiving the compound B, a meshed bar for the model rats for cognitive dysfunction and receiving physiologic saline, and a closed black bar for the model rats for cognitive dysfunction and receiving compound B. * represents significant difference.

FIG. 5 shows abnormal enhancement in latent inhibition observed in rats to which EGF was administered as neonates. The upper panel of this figure represents latent inhibition of control animals in rates of conditioned two-way active avoidance. The lower panel represents latent inhibition of the animal model for cognitive dysfunction in rates of conditioned two-way active avoidance.

Explanation of the marks; open circles represent scores of the pre-conditioned animals, closed circle represent scores of the animals without pre-conditioning. * represents significant difference.

FIG. 6 shows an ameliorative effect on abnormal latent inhibition induced by the pre-conditioning, by administration of the inhibitor for epidermal growth factor receptor activity. The upper panel of this figure represents effects of the inhibitor for epidermal growth factor receptor activity, Compound A, in control animals. The lower panel represents effects of the inhibitor for epidermal growth factor receptor activity, Compound A, in the animal model for cognitive dysfunction.

Explanation of the marks;

open circles for normal control rats receiving physiologic saline, closed circles for Compound A-injected animals.

FIG. 7 shows an ameliorative effect on abnormal latent inhibition induced by the pre-conditioning, by administration of the inhibitor for epidermal growth factor receptor activity. The upper panel of this figure represents effects of the inhibitor for epidermal growth factor receptor activity, Compound B, in control animals. The lower panel represents effects of the inhibitor for epidermal growth factor receptor activity, Compound B, in the animal model for cognitive dysfunction.

Explanation of the marks;

Circles represent rats receiving physiologic saline, squares represent Compound B-injected animals.

FIG. 8 shows an ameliorative effect on methamphetamine-induced hyperlocomotion, by administration of the inhibitor for epidermal growth factor receptor activity. The upper panel of this figure shows the total amount of vertical per hour, one hour after movement methamphetamine administration. The lower panel represents total horizontal locomotion.

Explanation of the marks;

Open circles represent normal control rats receiving Compound A. Closed circles represent the Compound A-injected rat model for cognitive dysfunction. * represents significant difference.

FIG. 9 shows decreased prepulse inhibition observed in rats to which PCP was administered as neonates. It is a control for FIG. 10 data.

Explanation of the marks;

Open bars for normal control rats (8 weeks old), closed bars for the rats for cognitive dysfunction (9 week old). * represents significant difference.

FIG. 10 shows an ameliorative effect on reduction of prepulse inhibition at 85 dB prepulse stimuli observed in rats to which PCP was administered as neonates, by administration of the inhibitor for epidermal growth factor receptor activity.

Explanation of the marks;

an open bar for normal control rats receiving physiologic saline, a closed black bar for control rats receiving Compound A, a dotted bar for the model rats for cognitive dysfunction receiving physiologic saline, and a meshed black bar for the PCP-induced model rats for cognitive dysfunction and receiving Compound A. * represents significant difference.

### DETAILED EXPLANATION OF THE INVENTION

Best Mode to Perform the Invention

Inhibitors for the activity of epidermal growth factor receptors represent the pharmaceutical agents that inhibit the activity of epidermal growth factor receptors in physiological condition. For example, these are the ligand neutralizing agents that bind to epidermal growth factor receptor to block their association with the receptor, the ligand binding blockers that directly act on the interactions of epidermal growth factor with their receptors, and the enzyme inhibitors for the tyrosine kinase of epidermal growth factor receptor, although not limited only to these compounds.

Derivatives of alpha-cyano-(3,4-dihydroxy)-cinnamic acid are known as agents inhibiting the tyrosine kinase enzyme of epidermal growth factor receptors. These compounds are thought to inhibit the activity of epidermal growth factors, preventing the ligands from binding epidermal growth factor receptors or decreasing the tyrosine kinase activity of the receptors (Ben-Bassat, H, et al.; Curr Pharm Des. 6: p 933-942 (2000)).

Derivatives of 4-phenylaminoquinazoline are well known as inhibitors for the activity of epidermal growth factor receptors. Recently, Gefiniti, which was approved in Japan in 2002 as an anti-cancer drug for lung tumor and become popular, is one of the quinazoline derivatives (Fry, D. W. Anti-Cancer Drug Design 15; p 3-16, (2000)).

Other agents include aeroplysinin-1 that is a derivative of natural bromotyrosine (Rodriguez-Nieto S. et al., FASEB J. p 261-263 (2002)) and 4-[(3-phenyl)amino]pyridopyrimidine that is a homologue of ATP (Smaill J. B., et al.; J. Med. Chem. 42; p 1803 (1999)). These compounds are thought to block the tyrosine kinase activity of epidermal growth factor receptors to inhibit the activity of epidermal growth factor receptors as well.

Including the quinazoline derivatives, the cinnamide derivatives, the tyrosine derivatives, and the pyridopyrimidine derivatives described above, the examples of the preferred inhibitors for epidermal growth factor receptor activity are the compounds listed below, modified compounds thereof, and pharmaceutically-acceptable acid salts thereof, however, they are not limited to only these compounds.
[(3,4-Dihidroxyphenyl)methylen]-propanedinitryl
[Gazit et al., Science 242; p 933 (1988)]
(E)-2-Cyano-3-(3,4-dihidroxyphenyl)-2-propenamide
[Yaish et al., Science 242; p 933 (1988)]
(E)-2-Cyano-3-(3,4-dihidroxyphenyl)-2-propenethioamide
[Yaish et al., Science 242; p 933 (1988)]
(E)-2-Cyano-3-{3,4-dihidroxyphenyl-N-(phenylmethyl)}-2-propenamide
[Gazit et al., J. Med. Chem. 34; p 1896 (1991)]
(E)-2-Cyano-3-(3,4-dihidroxyphenyl)-N-phenyl-2-propenamide
[Gazit et al., J. Med. Chem. 34; p 1896 (1991)]
(E)-2-Cyano-3-(3,4-dihidroxyphenyl)-N-(3-phenylpropyl)-2-propenamide
[Gazit et al., J. Med. Chem. 34: p 1896 (1991)]
(E)-2-Cyano3-(3,4-dihidroxyphenyl)-N-(3-phenylbutyl)-2-propenamide
[Gazit et al., J. Med. Chem. 34; p 1896 (1991)]
(E)-2-Cyano-3-(3,4-dihydroxyphenyl)-N-(1-phenylethyl)-2-propenamide
[Gazit et al., J. Med. Chem. 34; p 1896 (1991)]
(E)-(R)-2-Cyano-3-(3,4-dihydroxyphenyl)-N-(1-phenylethyl)-2-propenamide
[Gazit et al., J. Med. Chem. 34; p 1896 (1991)]
N-(3-chlorophenyl)-6,7-dimethoxy-4-quinazoline
[Levitzki and Gazit, Science 267; p 1783 (1995)]
4-(3-bromoanilino)-6,7-dimethoxyquinazoline
[Fry et al Science 265; p 1093 (1994)]
4-(3-chloro4-fluoroanilino)-7-methoxy-6-(3-morphorinopropoxy)quinazoline
[Gibson, K. H. et al.; Bioorganic Med. Chem. Lett. 7; p 2723 (1997)]
[4-(3-bromophenyl)anilino]-6,7-diaminoquinazoline
[Rewcastle, G. W. et al. J. Med. Chem. 39; p 918 (1996)]
{8-(3-bromophenyl)amino}-3-methyl-3H-imidazo[4,5γ]-quinazoline
[Rewcastle, G. W. et al. J. Med. Chem. 39; p 918 (1996)]
{8-(3-bromophenyl)amino}-1H-imidazo[4,5γ]-quinazoline
[Rewcastle, G. W. et al. J. Med. Chem. 39; p 918 (1996)]
{4-(3-bromophenyl)amino}-6,7-diethoxyquinazoline
[Bridges, A. J. et al. J. Med. Chem. 39; p 267 (1996)]
{4-(3-bromophenyl)amino}-6-acrylamidoquinazoline
[Fry, D. W. et al., Proc. Natl. Acad. Sci. USA 95; p 12022 (1998)]
{4-(3-bromopheny)amino}-6-propionylamidoquinazoline
[Fry, D. W. et al., Proc. Natl. Acad. Sci. USA 95; p 12022 (1998)]
((+)-Aeroplysinin-1) C9H9Br2NO3
[Koulman, A. et al. J. Nat. Prod. 59; p 591 (1996)]
4-[(3-bromophenyl)amino]-{6-methylaminopyrido}[4,5-e]pyrimidine
[Cunnick, J. M. et al. J. Biol. Chem. 273, p 14468 (1998)]
4-{3-chlorophenyl}amino}-{5,6-dimethyl-pyrrolo[3,4-e]}pyrimidine
[Traxler, P. M. et al., J. Med. Chem 39; p 2285 (1996)]
4-[(3-bromophenyl)amino]-{6-chloropropeonyl-pyrido}[3,4-e]}pyrimidine
[Smaill, J. B. et al., J. Med. Chem. 43: p 3199 (2000)]
4-[(3-bromophenyl)amino]-{6-ethylensulfino-pyrido}[4,5-e]}pyrimidine
[Smaill, J. B. et al., J. Med. Chem. 43: p 3199 (2000)]
α-Cyano-β-hydroxy-β-methyl-N-(2,5-dibromophenyl)propeneamide
[Mahajan, S., et al. J. Biol. Chem. 274; p 9587 (2000)]
{5-Amino{(N-2,5-dihydroxybenzyl)-N′-2-hydroxybenzyl}salicyclic acid; Lavendustin A
[Hu, D. E. and Fan, T. P., Br. J. Pharmacol. 114; p 262 (1995)]

In order to demonstrate whether inhibitors for the activity of epidermal growth factor receptors is effective on treatment of psychiatric disorder, it is appropriate to show that the above agents can ameliorate the psychiatric symptoms of the animal model for the psychiatric disorder.

There are several animal models for psychiatric disorders, schizophrenia and cognitive impairments, including a dizocilpine (MK-801)-inducing hyperlocomotion model and an apomorphine-inducing model exhibiting an abnormality in prepulse inhibition, however, the model animals are not limited only to these examples. The dizocilpine (MK-801)-inducing hyperlocomotion model exhibits an increase in locomotion as the symptom of psychiatric disorders. It is possible to verify the effectiveness of the inhibitor of the activity of epidermal growth factor receptors, by administrating the inhibitor of the activity of epidermal growth factor receptors to the animal model and showing suppression of the hyper-reactivity in locomotion in the model animal.

When inhibitors of the activity of epidermal growth factor receptors are used as a drug for the prevention or treatment of psychiatric disorders, these can be prepared as therapeutic drugs according to conventional drug formulation procedures and they can be administered to patients orally or non-orally.

For example, they can be formulated to tablet, capsule, elixir, microcapsule, sterile solution, emulsion solution, etc. Since the formulations prepared by these procedures are safe and low-toxic, they can be administered to human or warm-blooded animals (such as mice and rats). In the case of oral administration, doses of the relevant compounds or their salts in human adults (assumingly with 60 kg body weight) are approximately 0.1-1000 mg per day, more preferably 1.0-500 mg per day, further more preferably 50-200 mg per day, although they depend upon a targeted individual and a targeted psychiatric disorder. In the case of non-oral administration, assuming intervenous injection to normal size of human adults (60 kg), it is beneficial to intravenously inject approximately 0.01-300 mg, more preferably 0.1-200 mg, further more preferably 0.1-10 mg of the relevant compounds, daily. Administration to other animals can be performed with the equivalent doses calculated as 60 kg body weight.

Instead of orally administrating the medicine according to the present invention, it is possible to administer directly into the brain. The direct administration into the brain enables us to avoid the side-effects in whole body that have been observed in the conventional anti-cancer chemotherapy and to carry out the drug administration or treatment without considering the penetration of the agents through the blood-brain barrier. Intraventricular injection using an osmotic minipump or injection into the cerebrospinal fluid can be taken to perform the direct administration to the brain. For example, it will be beneficial to administer more than 5 mg per day of [4-(3-bromophenyl)anilino]-6,7-diaminoquianozoline (PD153035), calculating a dose based on human brain weight and its affinity to epidermal growth factor receptors (Ki=25 pM).

### EXAMPLES

Several experimental examples of this invention are explained in detail as follows. They are representative experiments of this invention, therefore, these examples should not be considered to limit the range of this invention.

### Example 1

According to the method written in Japanese Patent Application 2000-309042, EGF was administered subcutaneously to neonatal rats and the model rats that display various behavioral abnormalities similar to schizophrenic patients were prepared. Using this model animal, behavioral alterations were evaluated in the several tests that can commonly be applied to schizophrenic patients as well (Yasuyuki Shiiki, Toshihiko Morimoto, Molecular Psychiatry (Japanese) 1; p 369-399 (2001)).

This model animal displays various behavioral features, which can be monitored. For example, these included abnormal sensorimotor gating that is assessed as prepulse inhibition (PPI) of acoustic startle, impairment of social interaction behaviors that is measured by social interaction test, a change in memory persistency that is measured as latent inhibition scores and a decrease in working memory (Futamura, T. et al., Soc Neurosci. Abstr. 32; session No. 291.1 (2002)). Sotoyama, H. et al., Soc Neurosci. Abstr. 32; session No. 496.20 (2002)).

Using this animal model for cognitive/behavioral dysfunction, we performed the following experiments to examine whether the EGF receptor inhibitors are useful to ameliorate cognitive behavioral abnormalities.

(Experiment 1) Ameliorative Effects of EGF Receptor Inhibitors on PPI Abnormality

Newborn Sprague-Dawley (SD) rats were purchased from SLC (Shizuoka, Japan). Recombinant human EGF (Higeta-Syoyu Co, Chiba Japan) and cytochrome c (Sigma Chemical Co; control) were dissolved in physiologic saline. These agents were administered subcutaneously to rat pups on postnatal days 2, 4, 6, 8, and 10 at the nape of the neck at a dose of 1.75 μg of 1 g body weight).

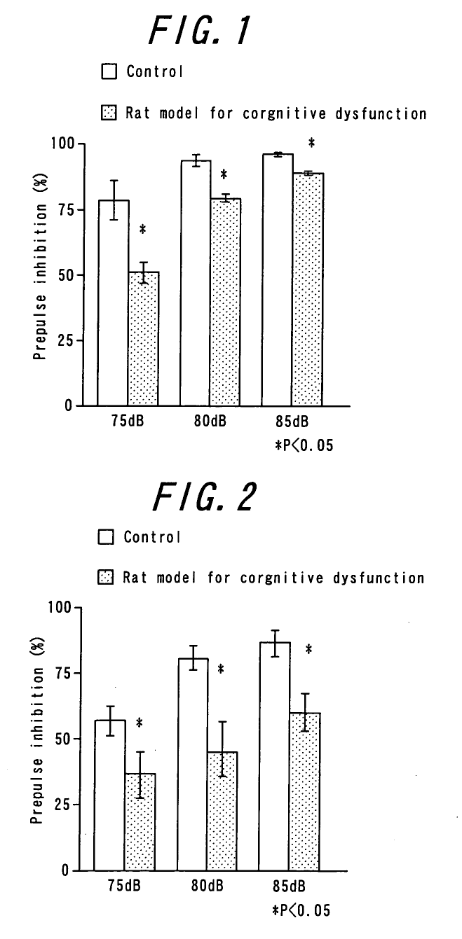

Acoustic startle amplitudes and PPI responses were measured in a startle reaction chamber for small animals (SR-Lab Systems, San Diego Instruments, San Diego, Calif.) from postnatal week 3. Acoustic startle was induced with acoustic stimuli (120 dB), in combination with three different prepulse intensities of 5-, 10-, and 15-dB-above background noise (75, 80, 85 dB). The 120 dB pulse was followed 100 ms after one of the prepulses was given. Each rat was placed in the startle chamber and initially acclimatized for 5 min with background noise alone. Prepulse inhibition (PPI) of a startle response was calculated as: 100−[(startle response on prepulse pulse stimulus trials−no stimulus trials)/(pulse alone trials−no stimulus trials)×100]. It is known that PPI responses are decreased in schizophrenic patients (Geyer, M. A. et al. Psychopharmacology (Berl), 156:p 117-154 (2001). ANOVA examination revealed that the EGF-treated group exhibited a decrease in PPI on postnatal week 8 (p<0.05, N=5) (FIG. 1 and FIG. 2).

SD rats treated with EGF or cytochrome c (control) were tested on postnatal days 56-66. [4-(3-bromophenyl)anilino]-6,7-diaminoquianozoline (referred as Compound A hearafter) and 4-(3-chloro4-fluoroanilino)-7-methoxy-6-(3-morphorinopropoxy) quianozoline (referred as Compound B hearafter) were dissolved in dimethylsulfoxide (DMSO) and diluted 10 times with saline before use. The same concentration of DMSO solution was used as a control.

After anesthesia, a 28 gauge cannula was inserted into the site of rat skull (0.3 mm anterior and 1.2 mm right lateral measured from the bregma, 4.5 mm below the skull) and glued to the skull with dental cement. The end of cannula was connected to an osmotic minipump (250 μl, effective for 14 days model 2002; Azla Corp.) via vinyl tubing. Pumps were implanted subcutaneously in the nape of the neck. Pumps had been filled with Compound A (1 mg/ml), Compound B (1 mg/ml) or the same concentration of DMSO (control). The scalp incision was closed with surgical suture and staples, and rats were maintained to wait recovery from the operation.

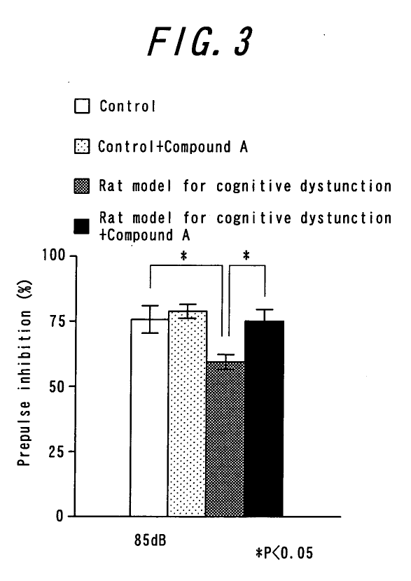

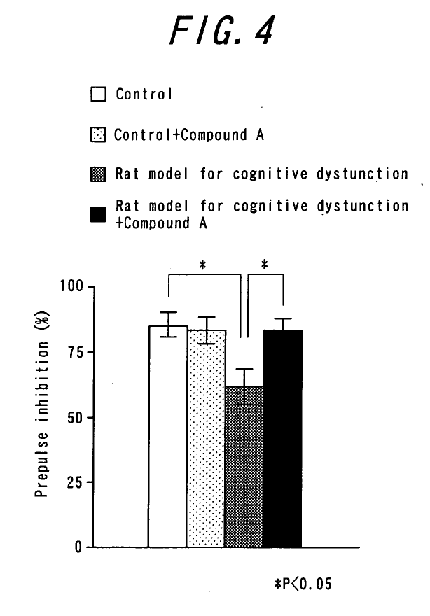

Seven days after administration, prepulse inhibition of the rats was monitored with 85 dB prepulse intensity using the acoustic startle measurement chamber for small animals (SR-Lab Systems, San Diego Instruments, San Diego, Calif.). The result showed that the intraventricular administration of Compound A and Compound B significantly ameliorated the abnormal decrease in prepulse inhibition in the model group for cognitive dysfunction, compared to the solvent-injected group (Compound A; P=0.011, N=5, Compound B; P=0.032, N=5). There were no significant differences between these model rats and control animals (Compound A; P=0.87, N=5, Compound B; P=0.54, N=5) (FIG. 3 and FIG. 4). Administration of Compound A and Compound B to normal control rats had no effects in prepulse inhibition. These results suggest that EGF receptor inhibitors are beneficial to ameliorate abnormal sensorimotor gating, which are observed in psychotic patients including schizophrenic patients as well.

### Example 2

(Experiment 2) Ameliorative Effects of EGF Receptor Inhibitors on Disruption of Latent Inhibition

The rat model for cognitive/behavioral dysfunction was prepared as EGF was administered to neonatal rats as described in the method of Experiment 1. SD rats were subjected to the two-way active avoidance task in an automated shuttle box (Muromachi-kiki, Tokyo, Japan) on postnatal weeks 6-8. The conditioned stimulus (CS) was an 80-dB tone and house light on and off. Rats learned the following task: When the CS was on, the animals had to cross to the other side of the shuttle box apparatus (avoidance response) in order to turn the CS off and avoid the appearance of the unconditioned stimulus (US). The US is an electric shock (0.6-mA, 10-sec), was given if the animal failed to make an escape response. Rats were given 6 sessions of two-way active-avoidance conditioning (10 trials per session)(total 60 trials). Active-avoidance learning was evaluated by scoring the number of avoidance rate to CS.

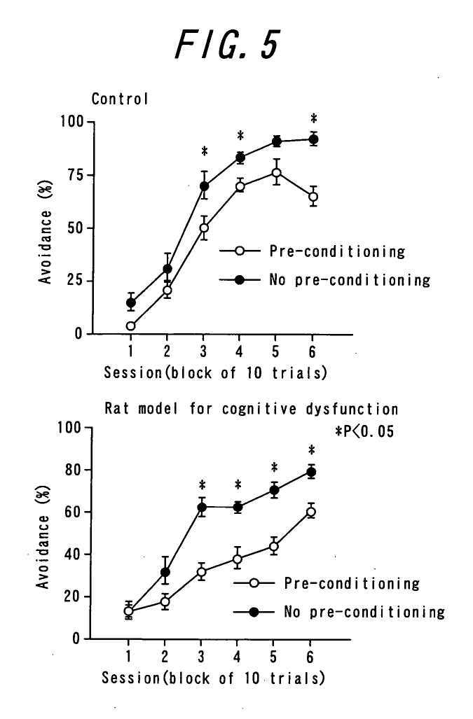

The rats for cognitive dysfunction that were prepared as described in Experiment 1 displayed normal learning ability in the present learning paradigm (FIG. 5 top; closed circle). This agreed with the previous reports (Futamura, T. et al., Soc Neurosci. Abstr. 32; session No. 291.1 (2002); Sotoyama, H. et al., Soc Neurosci. Abstr. 32; session No. 496.20 (2002)).

Before this learning test was conducted, the CS of an 80-dB tone and house light had been given to normal rats without delivering US (pre-conditioning), following avoidance learning was impaired in these rats (FIG. 5 top; open circle). This is latent inhibition that represents the effect of the pre-exposure of a conditioned stimulus (CS, e.g. tone), preventing the learning of unconditioned stimulus (Russig H et al. Neuropsychopharmacology 26: p 765-777 (2002)).

When the rat model for cognitive/behavioral dysfunction, which is described in the Experiment 1, was exposed to the pre-conditioning. In this model rats, learning of active avoidance in the novel conditioning session was more markedly inhibited than in control animals by pre-exposure. The pre-exposed rat model for cognitive dysfunction exhibited a more than 25% decrease in active avoidance rates after the 2nd session (FIG. 5 bottom; open circle). Generally, it is suggested that this phenomenon reflects abnormal persistency in animals. Such persistency can be inducible with hallucinogens such as cocaine (Murphy, C. A. et al. Behav Pharmacol. 12:p 13-23 (2001)).

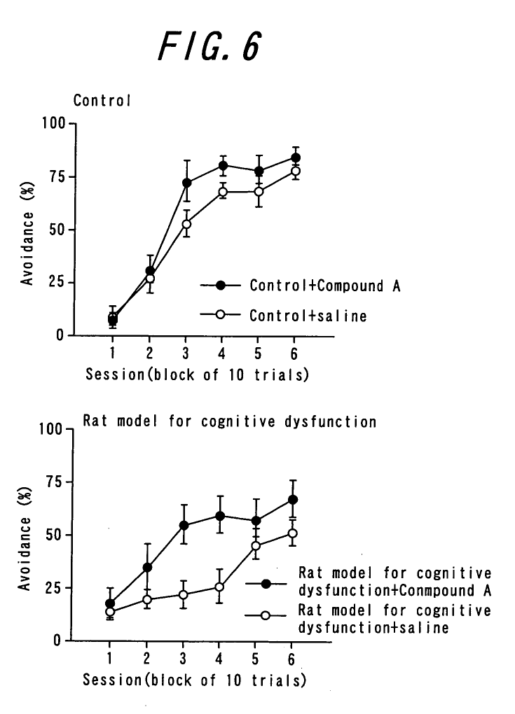

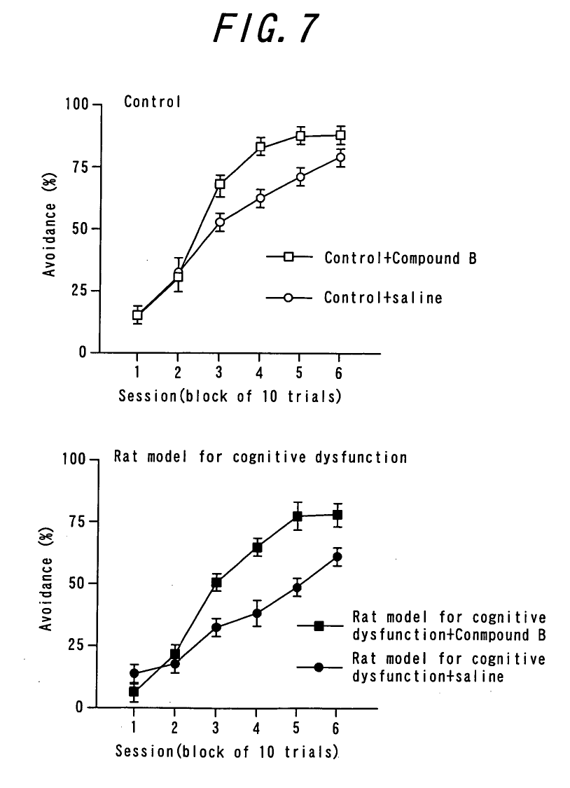

The EGF receptor inhibitors, Compound A and Compound B, were administered to the model rats for cognitive dysfunction or normal control rats according to the method described in Experiment 1. After administration both the normal control rats and the model rats for cognitive dysfunction exhibited better learning ability in conditioned avoidance learning (FIG. 6 and FIG. 7). Especially, EGF receptor inhibitors exerted a remarkable amelioration in latent inhibition of the model rats for cognitive dysfunction that showed a stronger impairment of latent inhibition in the two-way active avoidance paradigm (FIG. 6 bottom; closed circle, FIG. 7 bottom; closed square). Accordingly, their learning ability became indistinguishable from that of normal controls (FIG. 6 top; open circle, FIG. 7 top; open square). The results verifies that Compound A and Compound B significantly exert an ameliorative effect on abnormal increase in latent inhibition, which had been seen in the rat model for cognitive dysfunction (Compound A; P=0.018, N=5, Compound B; P=0.032, N=5) (FIG. 6 and FIG. 7). These results suggest that EGF receptor inhibitors are effective on ameliorating the symptoms of cognitive disorganization found in psychotic patients including schizophrenic patients.

### Example 3

(Experiment 3) Ameliorative Effects of an EGF Receptor Inhibitor on Enhancement of Locomotor Activity Following Repeated Methamphetamine Administration

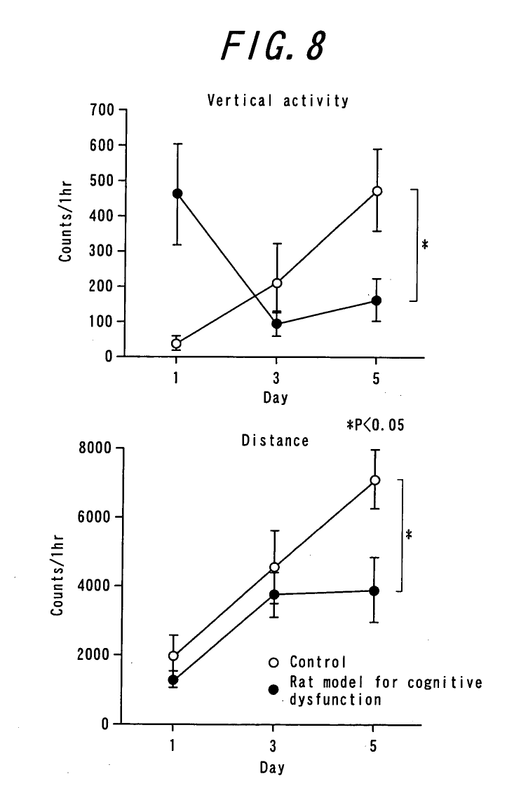

The model animals for cognitive dysfunction were prepared by administering EGF to neonatal rats as described in Experiment 1. Six weeks after the final EGF injection, the model rats for cognitive dysfunction and normal control rats were challenged with daily repeated injections of methamphetamine (2 mg/kg weight), which induces the symptoms of psychostimulant-induced psychosis. During the 5 continuous days of drug injections, locomotor activity, which involves dopaminergic function, was monitered 1 hr after each methamphetamine administration on days 1, 3, and 5. Rat were placed in an acrylic chamber (50 cm×50 cm) in a novel condition, and videotaped for 60 min. Total horizontal movement (FIG. 8, top) and total vertical activity (FIG. 8, bottom) were measured using an activity monitor (MED Associates, St. Albans, Va.).

There were no significant effects of intracerebroventricular infusion of Compound A into normal control rats, which was performed according to the method described in Experiment 1. Administration of methamphetamine generally increased both vertical movements and horizontal locomotion day by day and induced drug sensitization, which is so called psychostimulant poisoning symptom (open circle). When the model rats for cognitive dysfunction received the intracerebroventricular infusion of Compound A according to the method described in Experiment 1, the abnormal increase in locomotor activity (drug sensitization) that should be induced by repeated methamphetamine administration, however disappeared (close circle)(horizontal locomotion p=0.017; vertical activity p=0.022, N=5; T-test). These results indicate an EGF receptor inhibitor exerts an ameliorative effect on cognitive/behavioral impairments associated with schizophrenia.

### Example 4

According to the method of Wang C et al (Wang C et al., Neuroscience 107, 535-550, 2001), the animal model that exhibits the cognitive/behavioral abnormalities seen in schizophrenic patients was prepared by repeatedly administrating an N-methyl-D-aspartate receptor blocker, phencyclidine, to neonatal rats subcutaneously. In this model animal, various cognitive/behavioral performances were evaluated in several tests, which can be commonly applied to in schizophrenic patients as well (Yasuyuki Shiiki, Toshihiko Morimoto, Molecular Psychiatry (Japanese)1; p 369-399 (2001)).

This PCP-injected model animal displays various measurable features in behaviors. For example, these included abnormal sensorimotor gating that was evaluated with PPI of acoustic startle, impairment of social interaction behavior that was measured by social interaction test and an enhanced locomotor activity. Accordingly it is established that this is a model animal for schizophrenia (Semba J et al., Synapse 40, 11-18, (2001)).

Using this animal model for schizophrenia, it was examined whether the EGF receptor inhibitors are commonly effective to ameliorate the behavioral abnormalities of other animal models for schizophrenia.

(Experiment 4) Ameliorative Effect of EGF Receptor Inhibitors in Prepulse Inhibition Abnormality

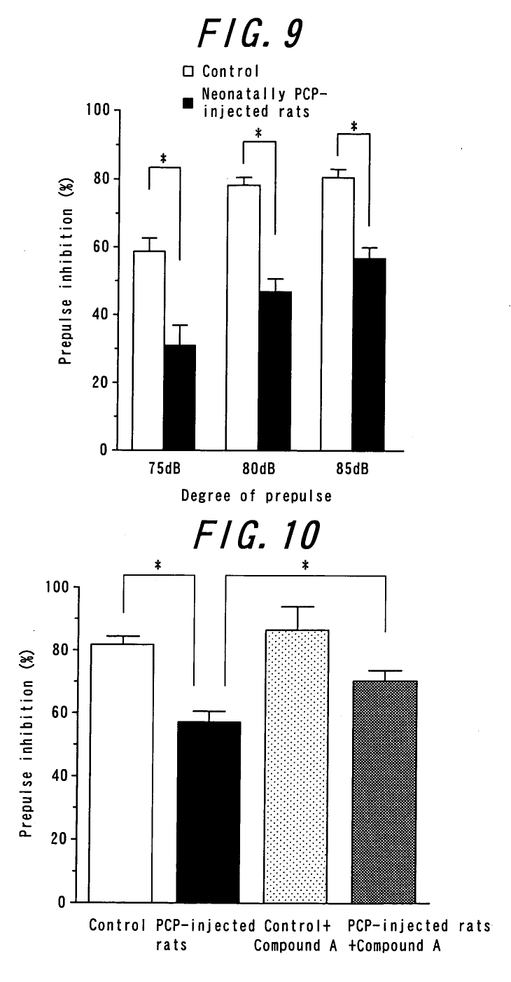

Newborn SD rats were purchased from SLC (Shizuoka, Japan). PCP and saline as controls were used. PCP was administered subcutaneously to rat pups on postnatal days 2, 4, 6, 8, 10, 12 and 14 (total 7 times) at the nape of the neck at a dose of 10 μg of 1 g body weight. Acoustic startle amplitudes and prepulse inhibition responses were measured in an acoustic startle chamber for small animals (SR-Lab Systems, San Diego Instruments, San Diego, Calif.) from postnatal weeks 3. Startle responses were induced with acoustic stimuli (120 dB) alone. The strengths of prepulse stimuli were 5, 10, or 15 dB above background noise (ie, 75-, 80-, or 85-dB prepulse, respectively). The main 120 dB pulse was followed 100 msec after one of the prepulses was given. Prepulse inhibition (PPI) of a startle response was calculated as: 100−[(startle response on prepulse pulse stimulus trials−no stimulus trials)/(pulse alone trials−no stimulus trials)×100]. It is known that PPI responses are decreased in schizophrenic patients (Geyer, M. A. et al. Psychopharmacology (Berl), 156:p 117-154 (2001). ANOVA examination indicates that neonatally PCP-treated rats had a decrease in prepulse inhibition on postnatal week 8 (p<0.05, N=5) (FIG. 9).

Neonatally PCP- or cytochrome c (control)-treated SD rats (Nippon SLC, Shizuoka, Japan) were tested on postnatal days 56-66. [4-(3-bromophenyl)anilino]-6,7-diaminoquianozoline (PD153035; referred as Compound A) was dissolved in DMSO and diluted 10 times by saline before use. The same concentration of DMSO solution was used as a control. A 28 gauge cannula was inserted into the skull of anesthetized rats, 0.3 mm anterior and 1.2 mm right lateral measured from the bregma, 4.5 mm below the skull and glued to the skull with dental cement. The end of cannula was connected to an Azlet osmotic minipump (250 μl, effective for 14 days model 2002; Azla Corp.) via vinyl tubing. Pumps were implanted subcutaneously in the nape of the neck. Pumps had been filled with Compound A (1 mg/ml) or the same concentration of DMSO solution. The scalp incision was closed with suture and surgical staples, and rats waited recovery from the operation.

Seven days after the initiation of the injection, PPI of the rats were examined with 85 dB prepulse intensities in acoustic startle measurement chamber for small animals (SR-Lab Systems, San Diego Instruments, San Diego, Calif.). Compound A injection to the rat model for cognitive/behavioral dysfunction, which were produced with neonatal PCP treatment, exhibited significant ameliorative effects against their smaller levels of prepulse inhibition, compared to the scores of the Compound A-injected normal rats (P=0.011, N=5) (FIG. 10). Compound A injection to normal control rats had no effects in prepulse inhibition. These results suggest that EGF receptor inhibitors are effective to ameliorate abnormal sensorimotor gating not only in neonatally EGF-treated rats, but also more generally and widely in psychotic patients including schizophrenic patients.

### INDUSTRIAL APPLICABILITY

This invention demonstrates that EGF receptor inhibitors have ameliorative effects on psychotic symptoms such as schizophrenia and provides novel antipsychotic drugs for the prevention and treatment of schizophrenia. Accordingly, this invention is applicable to the treatment of schizophrenia.

This application was filed with priority based on Japanese Patent Application 2003-34396.

1. A preventive and/or therapeutic agent for psychosis containing an inhibitor of epidermal growth factor receptor as the active ingredient.

2. The preventive and/or therapeutic agent according to claim 1, wherein the inhibition is a competitive inhibition on binding between epidermal growth factor receptor and epidermal growth factor.

3. A preventive and/or therapeutic agent for schizophrenia containing an inhibitor of epidermal growth factor receptor as the active ingredient.

4. The preventive and/or therapeutic agent according to claim 3, wherein the inhibition is a competitive inhibition on binding between epidermal growth factor receptor and epidermal growth factor.

5. A preventive and/or therapeutic agent for cognitive abnormalities containing an inhibitor of epidermal growth factor receptor as the active ingredient.

6. The preventive and/or therapeutic agent according to claim 5, wherein the inhibition is a competitive inhibition on binding between epidermal growth factor receptor and epidermal growth factor.

7. The preventive and/or therapeutic agent according to claim 1 containing a quinazoline derivative having inhibitory activity to epidermal growth factor receptor represented by the chemical formula I, a stereoisomer thereof, a pharmaceutically-acceptable salt thereof, its hydrate or its solvate as the effective ingredient, 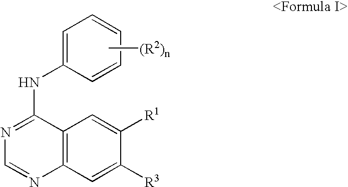
wherein n is 1, 2 or 3 and R² is each independently halogen, trifluoromethyl, or (1-4C)alkoxy; R³ is (1-4C)alkoxy; and R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy, and,
wherein any of the above-mentioned R¹ substituents comprising a CH₂ (methylene) group which is not attached to N or O atom optionally bears a hydroxy substituent on said CH₂ group.

8. The preventive and/or therapeutic agent according to claim 1 containing a quinazoline derivative represented by the chemical formula II, a stereoisomer thereof, a pharmaceutically-acceptable salt thereof, its hydrate or its solvate as the effective ingredient, 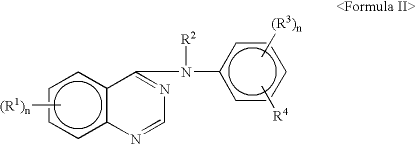
wherein; m is 1, 2, or 3; R¹ is each independently selected from the group consisting of hydrogen, halo, hydroxy, amino, hydroxyamino, carboxy, (C₁-C₄)alkoxycarbonyl, nitro, guanidino, ureido, carbarnoyl, cyano, trifluoromethyl, (R⁶)₂N-carbonyl, and phenyl-W-alkyl (wherein W is selected from the group consisting of a single bond, O, S and NH); or R¹ is each independently selected from the group consisting of cyano-(C₁-C₄)-alkyl and R⁹ (wherein R⁹ is selected from the group consisting of R⁵, R⁵O, (R⁶)₂N, R⁷C(═O), R⁵ONH, A and R⁵Y; R⁵ is (C1-C4 )alkyl; R⁶ is hydrogen or R⁵ wherein the R⁵s are the same or different; R⁷ is R⁵, R⁵0 or (R⁶)₂N; A is selected from the group consisting of piperidino-, morpholino, pyrrolidino and 4-R⁶-piperazin-1-yl, imidazol-1-yl, 4-pyridon-1-yl, carboxy-(C₁-C₄)-alkyl, phenoxy, phenyl, phenylsulfanyl, (C₂-C₄)-alkenyl, (R⁶)₂—N-carbonyl-(C1-C4)-alkyl; and Y is selected from the group consisting of S, SO, SO₂; the alkyl moieties in R⁵, R⁵O and (R⁶)₂N are halo or R⁹ (wherein R⁹ is defined as above) and wherein the resulting groups are optionally substituted with halo or R⁹, with the proviso that a nitrogen, oxygen or sulfur atom and another heteroatom can not be attached to the same carbon atom, and with the further proviso that no more than three “R⁹” units may comprise R¹; or each R¹ is each independently selected from the group consisting of R⁵-sulfonylamono, phthalimido-(C1-C4)-alkylsulfonylamino, benzamido, benzenesulfonylamino, 3-phenylureido, 2-oxopyrrolidin-1-yl, 2,5-dioxopyrrolidin-1-yl, and R¹⁰-(C₂-C₄)-alkanoylamino (wherein R¹⁰ is selected from halo, R⁶O, (C₂-C₄)-alkanoyloxy, R⁷C(═O), and (R⁵)₂N; and wherein said benzamido or benzenesulfonylamino or phenyl or phenoxy or anilino or phenylsulfanyl substituent in R¹ may optionally bear one or two halogens, (C₁-C₄)alkyl, cyano, methansulfonyl or (C₁-C₄)— alkoxy substituents); or any two R¹s taken together with the carbons to which they are attached may comprise a 5-8 membered ring comprising at least one or two heteroatoms selected from oxygen, sulfur or nitrogen; and wherein the alkyl groups and alkyl portions of the alkoxy or alkylamino groups may be straight chained or if comprised of at least three carbons may be branched or cyclic; R² is selected from hydrogen and optionally substituted(C₁-C₆)-alkyl; n is 1 or 2 and each R³ is independently selected from hydrogen, optionally substituted (C₁-C₆)-alkyl, optionally substituted amino, halo, hydroxy, optionally substituted hydroxy; R⁴ is azido or R¹¹-ethynyl (wherein R¹¹ is selected from hydrogen, optionally substituted(C₁-C₆)alkyl, wherein the substituents are selected from the group consisting of hydrogen, amino, hydroxy, R⁵0, R⁵NH and (R⁵)₂N.

9. The preventive and/or therapeutic agent according to claim 1 containing a quinazoline derivative having inhibitory activity to epidermal growth factor receptor represented by the chemical formula III, a stereoisomer thereof, a pharmaceutically-acceptable salt thereof, its hydrate or its solvate as the effective ingredient, 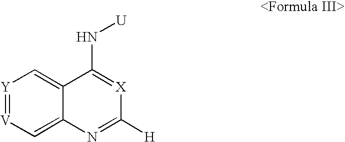
wherein X is N or CH; Y is CR¹ and V is N; or Y is N and V is CR¹; or Y is CR¹ and V is CR²; or Y is CR² and V is CR¹; R¹ represents a group CH₃SO₂CH₂CH₂NHCH₂—Ar—, (wherein Ar is selected from the group consisting of phenyl, furan, thiophene, pyrrole and thiazole, each of which may optionally be substituted by one or two halo, C₁₋₄ alkyl or C₁₋₄ alkoxy groups); R² is selected from the group consisting of hydrogen, halo, hydroxy, C₁₋₄ alkyl, C₁₋₄ alkoxy, C₁₋₄ alkylamino and di[C₁₋₄ alkyl]amino; U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl and 1H-benzotriazolyl group, substituted by an R³ group and optionally substituted by at least one R⁴ group selected independently; R³ is selected from a group consisting of benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl; or R³ represents trihalomethylbenzyl or trihalomethylbenzyloxy; or R³ represents a group of formula IV 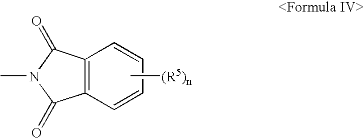
wherein each R⁵ is independently selected from the group consisting of halogen, C₁₋₄ alkyl and C₁₋₄ alkoxy; and n is 0 to 3; each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; with the proviso that the following compounds and their hydrochloride salts are excluded:
(1-Benzyl-1H-indazol-5-yl)-(6-(5-((2-methanesulphonyl-ethylamino)-methyl)-furan-2-yl)-pyrido[3,4-d]pyrimidin-4-yl-amine;
(4-Benzyloxy-phenyl)-(6-(5-((2-methanesulphonyl-ethylamino)-methyl)-furan-2-yl)-pyrido[3,4-d]pyrimidin-4-yl-amine;
(1-Benzyl-1H-indazol-5-yl)-(6-(5-((2-methanesulphonyl-ethylamino)-methyl)-furan-2-yl)-quinazolin-4-yl-amine;
(1-Benzyl-1H-indazol-5-yl)-(7-(5-((2-methanesulphonyl-ethylamino)-methyl)-furan-2-yl)-quinazolin-4-yl-amine;
(1-Benzyl-1H-indazol-5-yl)-(6-(5-((2-methanesulphonyl-ethylamino)-methyl)-1-methyl-pyrrol-2-yl)-quinazolin-4-yl-amine).

10. The preventive and/or therapeutic agent according to claim 1 containing a quinazoline derivative having inhibitory activity to epidermal growth factor receptor represented by the chemical formula V, a stereoisomer thereof, a pharmaceutically-acceptable salt thereof, its hydrate or its solvate as the effective ingredient, 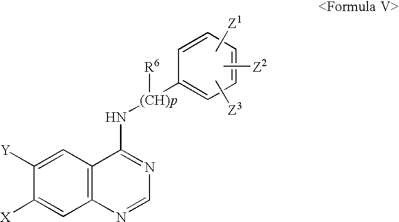
wherein
X is -D-E-F and Y is —SR⁴, —OR⁴, —NHR³, or hydrogen, or X is —SR⁴, —OR⁴, —NHR³, or hydrogen, and Y is -D-E-F;
D is NR²—, —O—, —CHR²—, —NR²—NH—, —NR²—O—, —CHR²—O—, —CHR²—CH₂—, —CHR²—CH₂—, NH—CHR²—, —O═CHR²—, —S—CHR²—, or D does not exist;
E is —CO—, —SO₂—, —PO(OR²)—, or —SO—;
F is —CR¹═CHR⁵—, —C≡C—R⁵—, —CR¹═C═CHR⁵;
with the proviso that when E is —SO— or —SO₂—, D is not —NH—CHR²—, or —O═CHR²;
R¹ is hydrogen, halogen, or C₁-C₆ alkyl;
R², R³, and R⁴ are independently hydrogen, C₁-C₆ alkyl, —(CH₂)ₙ—N-piperidinyl, —(CH₂)ₙ—N-piperazinyl,
—(CH₂)ₙ—N₄-piperazinyl [N₄—(C₁-C₆)alkyl],
—(CH₂)ₙ—N-pyrrolidyl, —(CH₂)ₙ—N-pyridinyl,
—(CH₂)ₙ—N-imidazoyl, —(CH₂)ₙ-imidazoyl
—(CH₂)ₙ—N-morpholino,
—(CH₂)ₙ—N-thiomorpholino,
—(CH₂)ₙ—N-hexahydroazepine or substituted C₁-C₆ alkyl,
wherein the substituents are selected from —OH, —NH₂, or —NA-B, A and B are independently hydrogen, C₁-C₆ alkyl, —(CH₂)ₙOH, —(CH₂)ₙ—N-piperidinyl, —(CH₂)ₙ—N-piperazinyl, —(CH₂)ₙ—N₁-piperazinyl[N₄—(C₁-C₆ alkyl)], —(CH₂)ₙ—N-pyrrolidyl, —(CH₂)ₙ—N-pyridyl, —(CH₂)ₙ-imidazoyl or —(CH₂)ₙ—N-imidazoyl; Z¹, Z², or Z³ are independently hydrogen, halogen, C₁-C₆ alkyl, C₃-C₈ cycloalkyl, C₁-C₆ alkoxy, C₃-C₈ cycloalkoxy, nitro, C₁-C₆ perfluoroalkyl, hydroxy, C₁-C₆ acyloxy, —NH₂, —NH(C₁-C₆ alkyl), —N(C₁-C₆ alkyl)₂, —NH(C₃-C₈ cycloalkyl), —N(C₃-C₈ cycloalkyl)₂, hydroxymethyl, C₁-C₆ acyl, cyano, azido, C₁-C₆ thioalkyl, C₁-C₆ sulfinylalkyl, C₁-C₆ sulfonylalkyl, C₃-C₈ thiocycloalkyl, C₃-C₈ sulfinylcycloalkyl, C₃-C₈ sulfonylcycloalkyl, mercapto, C₁-C₆ alkoxycarbonyl, C₃-C₈ cycloalkoxycarbonyl, C₂-C₄ alkenyl, C₄-C₈ cycloalkenyl, or C₂-C₄ alkynyl; and R⁵ is hydrogen, halogen, C₁-C₆-perfluoroalkyl, 1,1-difluoro(C₁-C₆)alkyl, C₁-C₆alkyl, —(CH₂)ₙ—N-piperidinyl, —(CH₂)ₙ-piperazinyl, —(CH₂)ₙ-piperazinyl[N₄-(C₁-C₆)alkyl], —(CH₂)ₙ—N-pyrrolidyl, —(CH₂)ₙ-pyridinyl, —(CH₂)ₙ—N-imidazoyl, —(CH₂)ₙ—N-morpholino, —(CH₂)ₙ—N-thiomorpholino, —CH═CH₂, —CH═CH—(C₁-C₆), N-hexahydroazepine, —(CH₂)ₙNH₂, —(CH₂)ₙNH(C₁-C₆ alkyl), —(CH₂)ₙ—N(C₁-C₆ alkyl)₂, -1-oxo(C₁-C₆)alkyl, carboxy, (C₁-C₆)alkyloxycarbonyl, N—(C₁-C₆)alkylcarbamoyl, phenyl or substituted phenyl, wherein the substituted phenyl may have from one to three substituents independently selected from Z¹, Z², Z³ or a monocyclic heteroaryl group, and each C₁-C₆ alkyl group may be substituted with —OH, —NH₂ or —NAB, (wherein and B are as defined above), R⁶ is hydrogen or C₁-C₆ alkyl; and n is 1 to 4, p is 0 or 1.

11. The preventive and/or therapeutic agent according to claim 1 containing a compound having inhibitory activity to epidermal growth factor receptor represented by the chemical formula VI, a stereoisomer thereof, a pharmaceutically-acceptable salt thereof, its hydrate or its solvate as the effective ingredient, 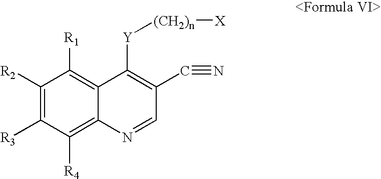
wherein X is cycloalkyl of 3 to 7 carbon atoms, which may be optionally substituted with one or more alkyl groups having 1 to 6 carbon atom; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; n is 0-1; Y is —NH—, —O—, —S—, or —NR—; R is alkyl of 1-6 carbon atoms; R₁, R₂, R₃, and R₄ are each independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 14 carbon atoms, N-alkylaminoalkyl of 2-7 carbon atoms, N,N-dialkylaminoalkyl of 3-14 carbon atoms, phenylamino, benzylamino, 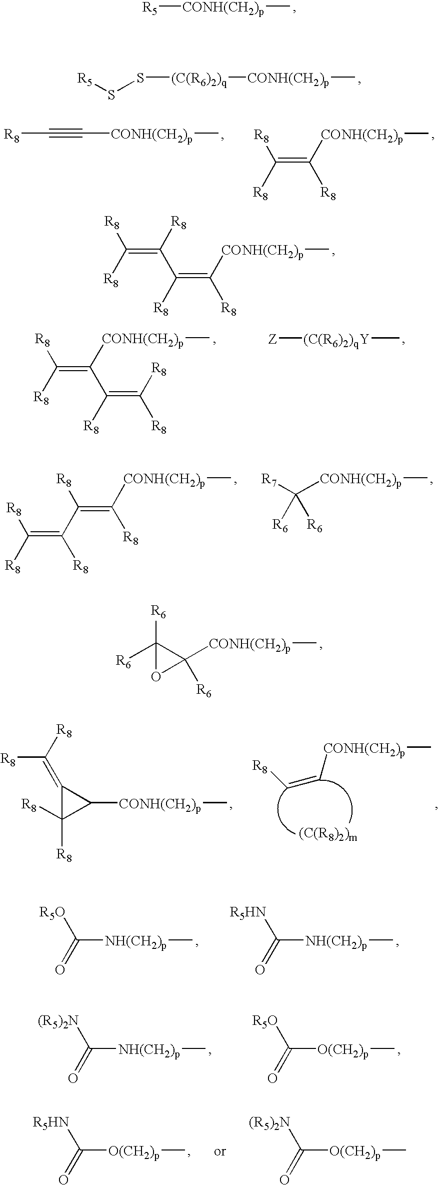
wherein, R₅ is alkyl of 1-6 carbon atoms, alkyl optionally substituted with one or more halogen atoms; phenyl, or phenyl optionally substituted with one or more halogen, alkoxy of 1-6 carbon atoms, trifluoromethyl, amino, nitro, cyano, or alkyl of 1-6 carbon atoms groups; R₆ is hydrogen, alkyl of 1-6 carbon atoms, or alkenyl of 2-6 carbon atoms; R₇ is chloro or bromo; R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl (wherein the alkyl group has 1-6 carbon atoms), piperidino-N-alkyl (wherein the alkyl group has 1-6 carbon atoms), N-alkyl-piperidino-N-alkyl (wherein either alkyl group has 1-6 carbon atoms), azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo; Z is amino, hydroxy, alkoxy of 1-6 carbon atoms, alkylamino (wherein the alkyl moiety has 1-6 carbon atoms), dialkylamino (wherein each of the alkyl moieties has 1-6 carbon atoms), morpholino, piperazino, N-alkylpiperazino (wherein the alkyl moiety has 1-6 carbon atoms), or pyrrolidino; m=1-4, q=1-3, and p=0-3; any of the substituents R₁, R₂, R₃, or R₄ that are located on contiguous carbon atoms may together be the divalent group —O—C(R₈)₂—O— (with the proviso that when Y is —NH—, R₁, R₂, R₃, and R₄ are hydrogen, and when n is 0, X is not 2-methylphenyl).

12. The preventive and/or therapeutic agent according to claim 1 containing a cinnamide derivative represented by the chemical formula VII, a stereoisomer thereof, a pharmaceutically-acceptable salt thereof, its hydrate or its solvate as the effective ingredient, 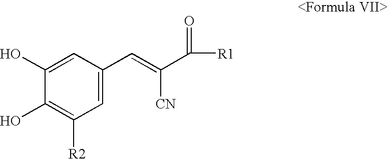
wherein R1 is preferably hydroxy, amino, alkylamino or phenyl amino group and R2 is preferably hydrogen, hydroxyl, nitro or t-butyl group.

13. The preventive and/or therapeutic agent according to claim 1 containing a pyridopyrimidine derivative represented by the chemical formula VIII, a stereoisomer thereof, a pharmaceutically-acceptable salt thereof, its hydrate or its solvate as the effective ingredient, 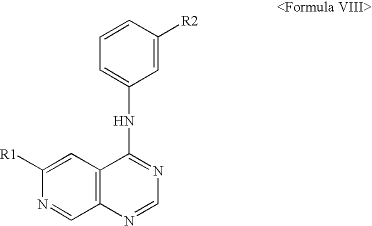
wherein R1 is preferably hydroxyl, amino, lower alkylamino, amide, alkylamide, alkenesulfinyl, or alkeneoxyamino group and R2 is preferably hydrogen or acetylene group.

14. The preventive and/or therapeutic agent according to claim 1 containing a tyrosine derivative represented by the chemical formula IX, a stereoisomer thereof, a pharmaceutically-acceptable salt thereof, its hydrate or its solvate as the effective ingredient, 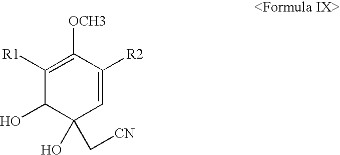
wherein R1 and R2 are preferably halogen atoms.

15. The preventive and/or therapeutic agent according to claim 1 containing 4-(3-chloro-4-fluoroanilino)-7-methoxy-6-(3-morpholinopropoxy)quinazoline, a stereoisomer thereof, a pharmaceutically-acceptable salt thereof, its hydrate or its solvate as the effective ingredient.

16. The preventive and/or therapeutic agent according to claim 1 containing {4-(3-bromophenyl)anilino}-6,7-diamino quinazoline, a stereoisomer thereof, a pharmaceutically-acceptable salt thereof, its hydrate or its solvate as the effective ingredient.
